(19) United States
(12) Patent Application Publication
(10) Pub. No.: US 2018/0008655 A1
(43) Pub. Date: Jan. 11, 2018

(54) PRESS DEVICE AND METHOD FOR PRODUCING RESINOUS PLANT EXTRACT

(71) Applicant: Kasse Weikel, Rackerby, CA (US)

(72) Inventor: Kasse Weikel, Rackerby, CA (US)

(21) Appl. No.: 15/084,029

(22) Filed: Mar. 29, 2016

(51) Int. Cl. A61K 36/185; B30B 9/04; B30B 15/34

(52) U.S. Cl. CPC A61K 36/185; B30B 15/34; B30B 9/047; A61K 2236/37

(57) ABSTRACT

The invention is a rosin press that is adapted for operation in two dimensions so that collection efficiency and amount of rosin from the device is maximized which allows for larger quantity operation and faster production. The press applies pressure to a quantity of resinous plant material placed between a pair of heated platens to initiate a flow of rosin. The press can be quickly and easily rotated from a first dimension to a second dimension so that the flow of rosin can fall by gravity to a chilled surface and be more easily collected while preserving quality. The invention can apply to any resinous plant.

### 1. RELATED APPLICATIONS

Not Applicable

### 2. TECHNICAL FIELD

This invention relates to devices and methods for producing extracts from resinous plant materials, and more specifically, to a device and method for placing a quantity of Cannabis under heat and pressure to produce a rosin extract.

### 3. BACKGROUND

Cannabis has long been used for medicinal and recreational purposes. The Cannabis plant contains resinous trichrome structures which are the source of cannabinoids like Tetrahydrocannabinol (THC) and Cannabidiol (CBD). Devices and methods of using Cannabis ideally seek to separate the trichromes to the greatest extent possible from the accompanying plant material. However, the plant material contains terpenoids and flavonoids, which provide desirable odor and flavor as well as a beneficial synergistic entourage effect. (See Ethan B. Russo, Taming THC: Potential Cannabis Synergy and Phytocannabinoid-Terpenoid Entourage Effects, British Journal of Pharmacology (2011) 163 1344-1364). The traditional method of using Cannabis has been to smoke the leaves and buds of the plant, but the combustion of the whole plant material introduces noxious components to the body in addition to desirable cannabinoids, terpenoids and flavonoids.

To maximize the production and preservation of the cannabinoid, terpenoid, and flavonoid components, while minimizing the amount of plant material, a number of extraction methods have arisen. Rosin refers to a resinous extract product created from an extraction process applied to Cannabis starting material that utilizes a combination of heat and pressure. With Cannabis, the heat and pressure method can either be used with flowers or less desirable leaves of the Cannabis plant and can also be used to clean up water hash and sifted kief into a full-melt hash oil of high purity. The result is a translucent, sappy, and sometimes clear and brittle glass-like product. If executed correctly, rosin can rival or surpass the flavor, potency, quality, and yield of other solvent-based extraction products with increased safety and ease. One reason for rosin's popularity is that it is a solvent-less technique, meaning that the process does not require the use of any volatile hydrocarbon solvents. Instead, rosin uses a mechanical process involving heat and pressure to extract the desired components from the plant.

Solvent extraction systems utilize light hydrocarbons such as butane and/or propane as solvents. Often, these complex and mechanical systems require a lengthy purge to safely remove most, if not all of the residual solvents from the final product. Rosin, on the other hand, simply uses heat and pressure and does not require any additional processing so the final product is clean and ready in minutes. When compared to butane hash oil (BHO), the two can be aesthetically indistinguishable. Rosin, when made properly, retains just as many if not more valuable terpenoids and flavonoids that account for aroma and flavor as well as clinically proven synergistic entourage effects. However, in a lab test, rosin will not contain residual hydrocarbons, thus giving it an “organic” quality.

Devices and methods for making rosin have included using a hair straightening iron to produce the heat, and pressure necessary for producing the rosin extract. In this method, the Cannabis starting material is usually contained between two pieces of parchment paper and the extracted rosin seeps onto the paper and can be scraped from the paper with a collection tool. This is a low-quantity production method that is practical for personal consumption but not for high-scale production.

For slightly increased yields and ease, Rosin Technologies, Inc. (www.rosintechnolgies.com) produces a pneumatic rosin press which uses heated platens to achieve rosin production. However, a drawback to this design is that if large quantities of Cannabis are processed, the rosin can reach a point of liquid flow where the liquid overwhelms the platens and flows indiscriminately in an undirected manner, making rosin collection messy and inefficient. Thus this previous device fails to maximize the rosin extracted from a large quantity of Cannabis. A Chinese firm by the name of Auplex (www.auplex.com) produces a similar pneumatic rosin press and therefore this vertical press design appears to presently be state of the art. However pneumatic presses are limited by expense, size, weight, and lower amounts of applicable force.

The present invention is a rosin press and method which employs heat and pressure to extract rosin from a large quantity of Cannabis. However the present invention addresses the drawbacks of the prior art with regard to maximizing the scale and ease of collection of rosin. The present invention addresses the constraints of prior art to process larger quantities of rosin. The present invention represents an advance in the art of rosin production for medicinal and recreational purposes.

The foregoing reflects the state of the art of which the inventor is aware, and is tendered with a view toward discharging the inventor's acknowledged duty of candor, which may be pertinent to the patentability of the present invention. It is respectfully stipulated, however, that the foregoing discussion does not teach or render obvious, singly or when considered in combination, the inventor's claimed invention.

### SUMMARY OF THE INVENTION

The invention is a rosin press which employs heat and pressure to produce rosin extract from a quantity of Cannabis or other resinous plants. The invention is also a method for producing rosin as well as a rosin product produced by the method. The inventive rosin press is adapted for operation in two dimensions so that collection of rosin from the device is maximized. During high-quantity production, the rosin can overflow the platens and without a way to contain and direct the overflow, high quantity production is problematic. The present invention addresses the overflow issue by being able to operate in two dimensions. In a first dimension, the inventive press is operated with the load contacting surfaces of the upper and lower platens placed horizontally, so that a larger quantity of Cannabis or other resinous plant material can be placed between the platens safely and easily. The Cannabis is preferably contained in a packet of parchment paper or PTFE film with an open end to allow the free flow of rosin from the open end. The platens are closed together with a quantity of Cannabis compressed between the load contacting surfaces of the platens under several tons of mechanical pressure. A hydraulic pump is the preferred method of supplying the necessary force to the platens for high-quantity rosin production. The device adds heat to the platens and the combination of heat and several tons of mechanical pressure causes the rosin to begin flowing within a short period of time.

When the flow of rosin starts, the device can be quickly and easily rotated ninety degrees to a second dimension, where the platens are vertical and the open end of the parchment or PTFE packet is facing the ground. The rosin then flows gravitationally out from between the platens onto a cold collection surface placed beneath the flow of rosin. The cold collection surface solidifies the rosin, prevents further spreading, and preserves the quality of the product. The cold collection surface could be a cold plate or a chilled scroll system. The inventive press and method results in easier collection of the rosin product and almost no loss. When one run of a quantity of Cannabis is completed the press can be rotated back to the first dimension to be reloaded quickly and easily with a quantity of Cannabis for another run. This process can be repeated indefinitely and is only limited by quantities of Cannabis material available and the physical limits of the press.

Further objects and advantages of the invention will be brought out in the following portions of the specification, wherein the detailed description is for the purpose of fully disclosing the preferred embodiments of the invention, without placing limitations thereon.

### BRIEF DESCRIPTION OF THE DRAWINGS

The invention will be more fully understood by reference to the following drawings which are for illustrative purposes only:

FIG. 1 is a front perspective view of the inventive press device.

FIG. 2 is a rear elevational view of the inventive press device.

FIG. 3 is an elevated front perspective view of the inventive press device coupled to a controller unit.

FIG. 4 is an exploded view of the press frame component and platen assemblies of the inventive press device.

FIG. 5 is a side view of the inventive press device

FIG. 6A is a perspective view of the packet which contains a quantity of resinous plant material.

FIG. 6B is an end view looking from the open side of the packet introduced in FIG. 6A.

FIG. 7A is a side view of the inventive press device shown resting in a first dimensional orientation.

FIG. 7B is a side view of the inventive press device of FIG. 7A shown rotated ninety degrees and resting on leg extensions in a second dimensional orientation; a flow of rosin end product is shown flowing onto a cold surface from between the first and second platens.

FIG. 8A shows the inventive press device being rolled into a second dimensional orientation by rolling on the angled edges of leg extensions.

FIG. 8B shows the completed roll of the device into a second dimension from its orientation introduced in FIG. 8A.

### DETAILED DESCRIPTION OF THE PREFERRED EMBODIMENTS

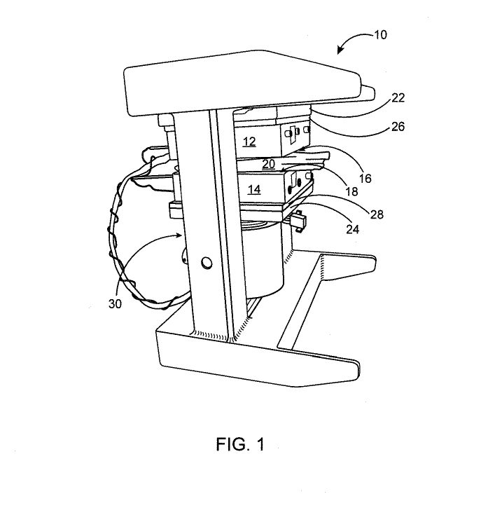

As shown in FIG. 1, the inventive press device 10 operates by applying two heated platens 12, 14 to a quantity of Cannabis, or other resinous plant material under several tons of mechanical pressure to create a flow of resinous plant extract. If Cannabis is used in production, the resinous plant extract is referred to as “rosin” and is an extract containing cannabinoids, terpenoinds, and flavonoids. The rosin extract flows in sufficient quantity to overflow the heated platens 12, 14, and this flow must be managed in order to derive a sufficient usable quantity in each production run. The present invention manages the flow of resinous plant extract by providing a press device 10 which can operate in a first dimension for safely and easily loading the device with plant material. In addition the press device 10 operates in a second dimension, which aids in managing and collecting the flow of resinous plant extract as it flows from between the heated platens 12, 14 undergoing mechanical pressure. The flow of rosin extract is collected on a cooled surface, such as a cold plate or scroll system. The temperature of the cold surface should be such that the flow of rosin is slowed or halted so that the product can be easily managed as well as preserving quality. To these ends, best results have been found at temperatures of between 30° F. to −20° F. Upon chilling, the rosin end-product can range from a sticky non-liquid consistency to a hard non-liquid brittle consistency.

The inventive device 10 is comprised of opposing first and second platens, 12, 14 wherein the platens have opposing load contacting surfaces 16, 18 which bear against the plant material in a leak-proof packet 20 during production of resinous plant extracts. The load contacting surfaces 16, 18 are preferably square in shape and of equal size on the two platens 12, 14. The platens rest indirectly on mounting platforms 22, 24 capable of enduring load forces of several tons up to over twenty tons. Preferably platforms 22, 24 are constructed of cold rolled steel at least ⅜″ in thickness. Heat barriers 26, 28 separate the platens 12, 14 from the mounting platforms 22, 24 and provides an indirect mounting of the platens to the mounting platforms, the heat barriers preventing the remaining portions of the press frame 30 and related components from becoming too hot to the touch and malfunctioning. The heat barrier is preferably constructed from Garolite G10/FR4. The platens 12, 14 are metallic, preferably made from aluminum to lighten the combined weight of the press device 10 as well as providing favorable thermal conductive properties.

The platens 12, 14 are heated in a controlled manner to achieve optimal temperature ranges for producing different characteristic in extracts. In the case of rosin production, there are time, pressure, and temperature profiles which are optimum for producing different types of rosin with certain characteristics. For example, the terpenoid and flavonoid components which gives rosin its desirable odor and flavor, is kept at its highest concentration at lower temperatures between 70° C.-100° C. Above 120° C. (220° F.) the quality of rosin decreases as terpenoids and flavonoids are volatilized and cannabinoids are decarboxylated in an increasing amount.

As noted, temperature affects the decarboxylation process of rosin. Decarboxylation is a chemical reaction that removes a carboxyl group and releases carbon dioxide (CO₂). With regard to Cannabis, decarboxylation must occur in order to release the full potential of the psychoactive effects of the plant. Δ9-Tetrahydrocannabinolic acid (THC-A) decarboxylates to give the psychoactive and medicinal compound Δ9-Tetrahydrocannabinol (THC). However, edible and topical applications of cannabis can provide non-psychoactive benefits if the THC-A component is emphasized. For example studies have supported THC-A as being an effective anti-inflammatory compound.

Decarboxylation occurs by heating Cannabis and hence the operator of the invention can control the heating profile to produce different rosin extract end products. With regard to the present invention, at lower temperatures, less decarboxylation occurs and at higher temperatures, greater decarboxylation occurs. The invention can therefore be used to achieve rosin products having a myriad of rosin production profiles which balance the flavor and synergistic component of the terpenoids along with the desired ratios of THC-A and THC of the rosin for recreational and/or medicinal purposes. The final physical state of material can also be manipulated with different amounts of heat leading to a “budder”, with a soft and porous structure or “shatter” exhibiting a brittle, glass-like texture.

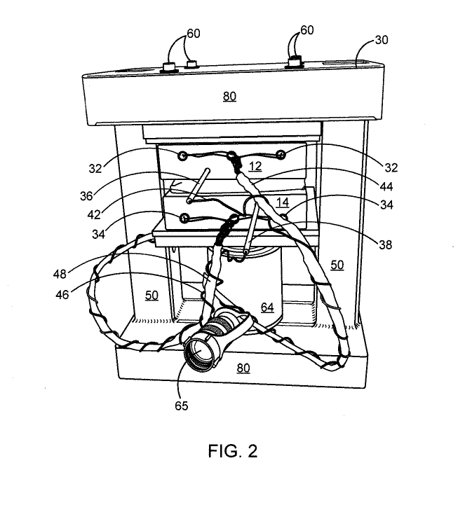

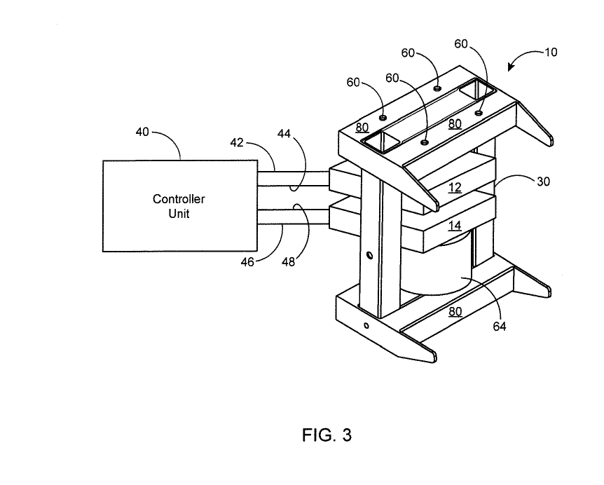

As shown in FIGS. 2 and 3, the first and second platens 12, 14 are heated by heat elements 32, 34 inserted into preferably both platens 12, 14. To these ends, heat elements 32, 34 are distributed in platens to provide an even distribution of heat throughout platens 12, 14. A temperature sensor 36, 38 is also preferably a component of both platens 12, 14, the temperature sensor communicating platen temperature data to a temperature controller unit 40. Temperature sensor can be a thermocouple, for example. The temperature controller unit is a computer which can be programmed to apply heat to the platens to achieve and hold certain temperatures. If certain rosin production profiles are desired, the temperature controller unit 40 can be programmed to achieve them. The main function of the temperature controller unit 40 is to maintain precise and accurate temperatures at load contacting surfaces 16, 18 desired by the operator. The temperature sensor 36 and heat elements 32 of the first platen 12 are connected to the controller by a temperature sensor line 42 and a heat element line 44. Similarly, second platen 14 is connected to controller 40 by temperature sensor line 46 and heat element line 48.

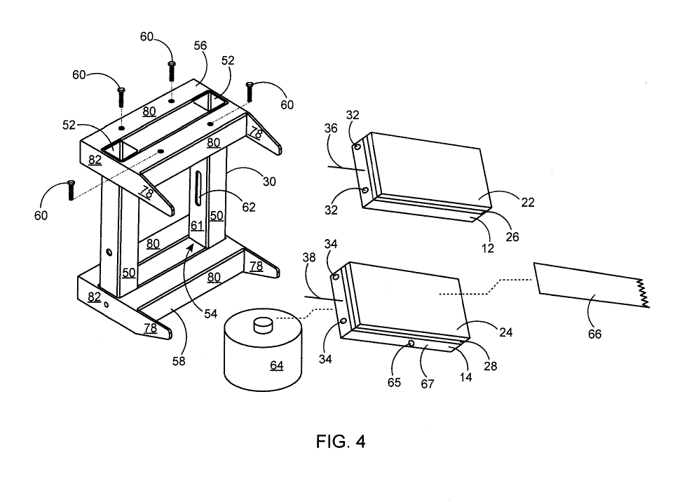

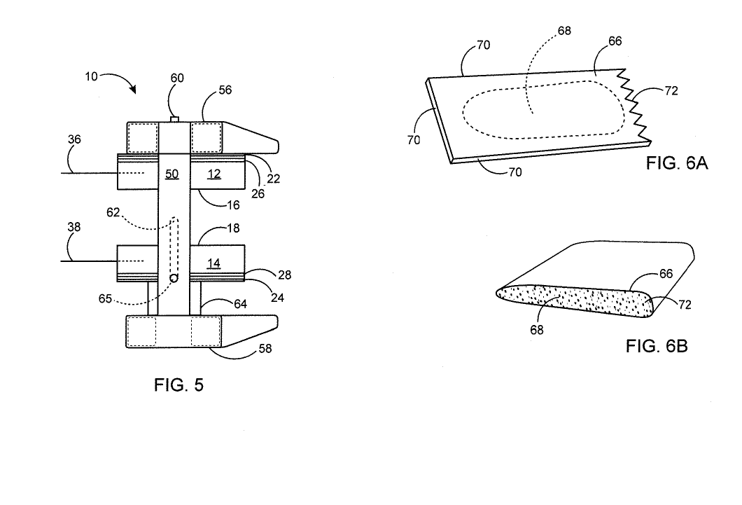

The platens 12, 14 are mounted in a press frame 30 as shown in FIG. 4 between twin opposing columns 50, the columns being spaced a distance apart. Each opposing column 50 has a first end 52 and a second end 54 and each first end 52 is connected to a first base end 56 and each second end 54 is connected to a second base end 58. The first platen 12 is mounted in a stationary position to first base end 56, while the second platen 14 is movably mounted to the opposing columns via vertical guide slots 62 and motion device 64. First platen 12, mounting platform, 22 and heat barrier 26 are mounted in a stationary position to first base end 56 with fasteners 60. As further shown in FIG. 5, to facilitate vertical movement along the columns 50, the second platen 14, mounting platform 24 and heat barrier 28 are mounted in vertical guide slots 62 located on the interior side 61 of columns 50. Platen 14 movement in vertical guide slots 62 is facilitated by roller bearings 65 mounted on each side 67 of platen 14. The movement of platen 14 is facilitated by a motion device 64 that is attached to the second platen 14 for vertically moving the load contacting surface 18 of the second platen 14 a distance to contact the load contacting surface 16 of the first platen 12. The motion device 64 closes the load contacting surfaces 16, 18 of the first platen 12 and the second platen 14 together and provides a mechanical closing force of preferably several tons to over twenty tons. A hydraulic ram cylinder with a load designation of twenty tons has been found to be able to apply sufficient mechanical force against a quantity of Cannabis to provide a sufficient flow of rosin without over-stressing the press frame 30. Hydraulic ram cylinder acting as a motion device 64 attaches to a hydraulic pump (not shown) at connection point 65.

The platens 12, 14 apply pressure to a quantity of Cannabis or other resinous plant material 68 that is placed in a packet 66 like that shown in FIG. 6A. The packet 66 can be made of parchment paper or PTFE film that is relatively leak proof. The packet is generally square or rectangular-shaped and is closed off on three sides 70, leaving an open side 72 to allow an exit for the flow of rosin. FIG. 6B shows an end view of packet 66, providing a view of the Cannabis starting material or other resinous plant starting material 68 located therein. Upon being placed between the platens 12, 14, the packet 66 is subjected to extreme force, exceeding twenty+ tons, while being heated. Due to the extreme force applied to packet 66, it is advisable in certain circumstances to insert packet 66 into an additional envelope of stainless steel mesh screen (not shown). Typically, the controlled application of heat and pressure starts the flow of rosin quickly.

The prior art is replete with rosin presses that operate heated platens and which mechanically press the platens together in a vertical direction. This configuration found in the prior art is convenient for loading the rosin press with a quantity of Cannabis or other resinous plant material. However, this configuration is inefficient when it comes to containing and managing a flow of rosin emitted from between the platens during a larger production run. The rosin tends to spread thin and can exhibit “fountain flow” which overwhelms the ability of an operator to contain and collect the rosin to any degree of efficiency.

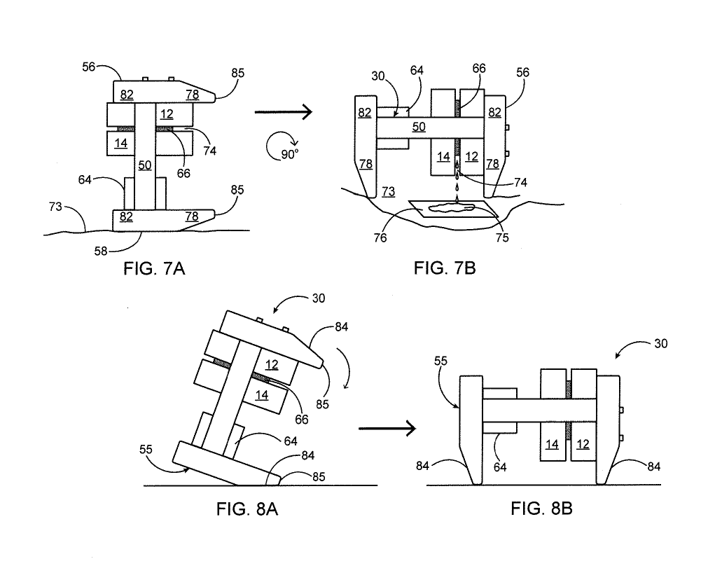

The present inventive rosin press 10 has solved the problem of efficient larger scale rosin extraction and collection by providing a rosin press 10 that can operate in a second dimension which aids in flow of rosin utilizing gravity. As shown in FIGS. 7A and 7B, the invention is rotated 90° from a first dimension (FIG. 7A) to a second dimension (FIG. 7B) so that the gap 74 between the first platen 12 and the second platen 14 is facing the ground 73 which allows the rosin 75 to flow by gravity onto a cooled surface 76, which can be a cold plate or a chilled conveyor or scroll surface. While the press 10 is oriented in its second dimension, the platens 12, 14 are kept elevated above the ground 73 during the flow of rosin 75 by leg extensions 78. Leg extensions 78 are attached to the base ends 56, 58 which are comprised of two cross pieces 80. The leg extensions 78 extend outwardly from, and are preferably integral with, end caps 82, which provide additional structural integrity to base ends 56, 58 by tying the cross pieces 80 of base ends 56, 58 and the columns 50 together. Leg extensions 78 have an angled edge 84 on all four leg extensions of the press frame 30. The angled edge 84 allows the press 10 to be rolled forward from the first dimension to the second dimension quickly and easily without having to lift the press. In the second dimension orientation as shown in FIGS. 7B and 8B, the press device 10 rests on the ends 85 of leg extensions. The angled edge 84 allows the transition between dimensions to occur by quickly and easily rolling the device, rather than having to lift and reposition the device, as shown in FIGS. 8A and 8B. Angled edge 84 is adjacent to the resting face 55 of base end 58 and allows the device 10 to roll forward, transitioning from resting face 55 to angled edge 84 and finally to a fully ninety-degree change in orientation as shown. Once the rosin extraction and collection is completed, the device 10 can be rolled back to the first dimension with relative ease.

The fact of being able to quickly and easily rotate from a first dimension to a second dimension gives the inventive rosin press advantages in efficiently extracting and collecting a larger quantity of rosin. It has been found that the transition to the second dimension with accompanying collection of the flow of rosin increases production at between five to ten times the quantity or more of that experienced by the rosin presses of the prior art. The transition to the second dimension provides a gravitational component in aiding the flow of rosin or other resinous plant extracts that is not achieved by the prior art. The transition to the second dimension also provides a large and unobstructed area for chilled surfaces to be inserted beneath the gap in the platens and used for collecting larger amounts of rosin in a substantially more efficient manner which also preserves the quality of the rosin.

Finally, although the description above contains much specificity, this should not be construed as limiting the scope of the invention, but as merely providing illustrations of some of the presently preferred embodiments of this invention. This invention may be altered and rearranged in numerous ways by one skilled in the art without departing from the coverage of any patent claims, which are supported by this specification.

1. A press for producing a fluid extract from resinous plants, the press comprising:
a first platen with a load contacting surface, said first platen further comprising a heat source;
a second platen with a load contacting surface, said second platen further comprising a heat source;
a controller, said controller communicating with said heat source of said first platen and said heat source of said second platen, said controller regulating an amount of heat introduced into said heat sources of said first platen and said second platen;
at least one temperature sensor in communication with at least a one of said platens, said temperature sensor communicating temperature data to said controller;
a motion device for closing together said load contacting surfaces of said first and said second platens;
a press frame comprising opposing columns, said opposing columns of said press frame being spaced a distance apart to accommodate said first and said second platens there between;
said opposing columns each comprising first and second ends;
said press frame further comprising opposing first and second base ends, wherein said first base end is attached to said first ends of said opposing columns and said second base end is attached to said second ends of said opposing columns;
said first and second base ends each further comprising leg extensions, said leg extensions protruding outwardly from said first and second base ends of said press frame;
wherein said press can be rotated from a first dimension resting upon a one of said base ends to a second dimension resting upon said leg extensions.

2. The press as recited in claim 1, wherein said leg extensions further comprise an angled edge closest to a resting face of at least a one of said base ends.

3. The press as recited in claim 1, wherein said columns have an inboard side comprising a vertical guide slot for mounting said second platen, said vertical guide slot allowing said second platen mounted therein to travel vertically toward said first platen.

4. The press as recited in claim 3, wherein said second platen is moved vertically in said slot by said motion device for closing together said load contacting faces of said first and said second platens.

5. A press for producing a fluid extract from resinous plants, the press comprising:
a first platen with a load contacting surface;
a second platen with a load contacting surface;
a heat source for heating at least a one of said first platen or said second platen;
a controller, said controller communicating with said heat source, said controller regulating an amount of heat introduced into said heat source;
at least one temperature sensor in communication with at least a one of said first platen or second platen, said temperature sensor communicating temperature data to said controller;
a motion device for closing together said load contacting surfaces of said first and said second platens;
a press frame comprising opposing columns, said opposing columns of said press frame being spaced a distance apart to accommodate said first and said second platens there between;
said opposing columns each comprising first and second ends;
said press frame further comprising opposing first and second base ends, wherein said first base end is attached to said first ends of said opposing columns and said second base end is attached to said second ends of said opposing columns;
said first and second base ends each further comprising leg extensions, said leg extensions protruding outwardly from said first and second base ends of said press frame;
wherein said press can be rotated from a first dimension resting upon a one of said base ends to a second dimension resting upon said leg extensions.

6. A press for producing a fluid extract from resinous plants wherein said press comprises first and second platens for applying heat and pressure to a quantity of resinous plant material, said press further transitioning from a first dimension to a second dimension, wherein said second dimension orients said platens for capturing a free flow of fluid extract flowing from between said platens.

7. The press for producing a fluid extract as recited in claim 6, wherein said press is supported on a base end in said first dimension.

8. The press for producing a fluid extract as recited in claim 7, wherein said press is supported on a plurality of leg extensions in said second dimension.

9. The press for producing a fluid extract as recited in claim 6, wherein said fluid extract is collected on a cold surface.

10. A fluid extract produced by the press recited in claim 6.

11. A process for producing a fluid extract from plant material, the process comprising:
a) inserting a quantity of plant material between a pair of platens mounted in a press frame, wherein at least a one of said platens is heated;
b) closing said platens together to apply mechanical pressure and heat to said plant material;
c) rotating said press frame so as to orient said press frame in a direction such that a gap between said platens is pointed toward a ground surface; and
d) capturing a flow of fluid extract upon a chilled surface positioned below said gap between said platens.
